(19) United States
(12) Patent Application Publication
(10) Pub. No.: US 2021/0236712 A1
(43) Pub. Date: Aug. 5, 2021

(54) Vortical Ear Irrigation

(71) Applicant: Veartech, Inc., Seguin, TX (US)

(72) Inventors: John Rheiner, New Braunfels, TX (US); Terry Young, La Vernia, TX (US)

(73) Assignee: Veartech, Inc., Seguin, TX (US)

(21) Appl. No.: 16/781,628

(22) Filed: Feb. 4, 2020

(51) Int. Cl. A61M 3/02; A61B 1/00; A61B 1/05; A61B 1/227

(52) U.S. Cl. CPC A61M 3/0283; A61M 3/0287; A61B 1/00195; A61B 1/05; A61B 5/01; A61B 1/227; A61B 1/00016; A61B 1/00103; A61M 2210/0662; A61B 1/00045

(57) ABSTRACT

There is disclosed in one example an ear irrigation system, including: a control unit including a fluid reservoir; a disposable earpiece constructed to interface with a human subject's ear canal; an extension unit configured to receive the disposable earpiece and to impart fluid to an ingress port of the disposable earpiece; and an umbilical to fluidly couple the fluid reservoir to the extension unit; wherein the control unit is programmed to, responsive to an input from an operator, pump fluid from the fluid reservoir to the extension unit; and wherein the disposable earpiece includes a plurality of vortical fluid guides to guide fluid from an ingress port to an egress port, and to impart to the fluid at the egress port a vortical motion to delaminate a cerumen impaction from the human user's ear canal.

### FIELD OF THE SPECIFICATION

This application relates in general to ear irrigation, and more particularly, though not exclusively, to a system and method of providing vortical ear irrigation.

### BACKGROUND

Buildup of earwax (known to medical professionals as cerumen impaction) is a common condition that can result in a variety of complications. Safe, effective removal of earwax may occasionally become necessary in both home and clinical office environments.

### BRIEF DESCRIPTION OF THE DRAWINGS

The present disclosure is best understood from the following detailed description when read with the accompanying FIGURES. It is emphasized that, in accordance with the standard practice in the industry, various features are not necessarily drawn to scale, and are used for illustration purposes only. Where a scale is shown, explicitly or implicitly, it provides only one illustrative example. In other embodiments, the dimensions of the various features may be arbitrarily increased or reduced for clarity of discussion. Furthermore, the various block diagrams illustrated herein disclose only one illustrative arrangement of logical elements. Those elements may be rearranged in different configurations, and elements shown in one block may, in appropriate circumstances, be moved to a different block or configuration.

FIG. 1A is a perspective view illustrating selected features of a cerumen removal system.

FIG. 1B is a perspective view illustrating a healthcare professional treating a patient.

FIG. 2 is a perspective view illustrating an embodiment of a handheld unit.

FIG. 3 is a perspective view illustration of an additional embodiment of a handheld unit.

FIGS. 4A, 4B, 4C, and 4D illustrate one embodiment of a disposable earpiece.

FIGS. 5A and 5B provide a perspective view of another embodiment of a disposable earpiece.

FIGS. 6A, 6B, 6C, 6D, 6E, and 6F illustrate yet another embodiment of a disposable earpiece.

FIGS. 7A and 7B are a perspective view illustration of a further embodiment of a disposable earpiece.

FIGS. 8 and 9 provide a block diagram illustration of another further embodiment of a disposable earpiece.

FIGS. 10A, 10B, 10C, and 10D illustrate an additional further embodiment of a disposable earpiece.

FIG. 11 is a block diagram illustration of a control unit.

FIG. 12 is a flowchart of a method of performing cerumen impaction removal with a cerumen removal system of the present specification.

### SUMMARY

In an example, there is disclosed an ear irrigation system, comprising: a control unit comprising a fluid reservoir; a disposable earpiece constructed to interface with a human subject's ear canal; an extension unit configured to receive the disposable earpiece and to impart fluid to an ingress port of the disposable earpiece; and an umbilical to fluidly couple the fluid reservoir to the extension unit; wherein the control unit is programmed to, responsive to an input from an operator, pump fluid from the fluid reservoir to the extension unit; and wherein the disposable earpiece comprises a plurality of vortical fluid guides to guide fluid from an ingress port to an egress port, and to impart to the fluid at the egress port a vortical motion to delaminate a cerumen impaction from the human user's ear canal.

### EMBODIMENTS OF THE DISCLOSURE

The following disclosure provides many different embodiments, or examples, for implementing different features of the present disclosure. Specific examples of components and arrangements are described below to simplify the present disclosure. These are, of course, merely examples and are not intended to be limiting. Further, the present disclosure may repeat reference numerals and/or letters in the various examples. This repetition is for the purpose of simplicity and clarity and does not in itself dictate a relationship between the various embodiments and/or configurations discussed. Different embodiments may have different advantages, and no particular advantage is necessarily required of any embodiment.

The medical term for a buildup of wax in the ear is a cerumen impaction. Cerumen impaction is a very common condition that can be caused by infection, allergies, or simply the passage of time. While there are many known methods for removing a cerumen impaction, most of them are uncomfortable to the patient or user, and if not done carefully or correctly, can actually cause damage to the eardrum.

For example, consumers have long used cotton swabs to try to remove ear wax themselves. This is generally not advised by medical professionals for several reasons. First, when a person sticks a cotton swab in their ear, there is danger they could jam it in too far and damage the eardrum. Furthermore, the cotton fibers can mix with the cerumen and act as a cement binder. In that case, the use of a cotton swab could actually make the impaction worse than it was to begin with.

Ear, nose, and throat (ENT) doctors and other medical practitioners have apparatuses that can be used to remove an impaction. These can, for example, shoot a stream of water into the user's ear, and/or use suction to remove the impaction. These have disadvantages in that cold water can be uncomfortable for the patient, and the heavy suction required to pull out the impaction can also be uncomfortable for the patient. Furthermore, fluids that mismatch the body temperature can cause vertigo and/or associated nausea or vomiting.

In removing a cerumen impaction, it is beneficial to first delaminate the impaction from the ear canal. In other words, the earwax binds to the ear canal like an adhesive. This lamination makes the impaction more difficult to remove. However, if the impaction is first delaminated from the ear canal, it can be more easily sucked out. It can also be broken up with a stream of body temperature water before being removed, thus increasing the comfort to the patient.

A mechanism for delaminating the impaction from the ear canal includes the use of a vortical water stream. Embodiments of the present specification include a cerumen removal system or systems that operate on this principle. In one illustrative and nonlimiting embodiment, a control unit is connected to a handheld unit via an umbilical. Note that the control unit may be a handheld unit, or some other extension unit like a fixed attachment. The control unit may provide a water reservoir and regulate the water's temperature. The umbilical can provide both signals and water to the handheld unit.

An end user or a medical professional can hold the handheld unit up to the ear. The control unit imparts a stream of water at a selected pressure and velocity to the handheld unit. The handheld unit directs the water to an earpiece, which is optionally a disposable earpiece, that is used one time for the individual user.

The disposable earpiece can include one or more ingress ports for the water. The ingress ports direct the water to a fluid guide that imparts a helical motion to the water stream. The water stream exits the disposable earpiece at one or more egress ports with the described vortical motion. The vortical action of the fluid may be used to delaminate the impaction from the ear canal. Once the impaction is delaminated, it can be either sucked out with pneumatic suction, and/or broken up with a linear water stream.

Once the earwax is loosened, its exit from the ear canal may be aided by the egress of the injected water or fluid. The fluid and the earwax can be sucked out of the ear, it can be directed down a drain line to a disposal reservoir, where it can be properly disposed of as bio waste.

Embodiments of the cerumen removal system of the present specification may also provide other features. For example, a biometric attachment could be used to measure the user's present body temperature. A heater within the control unit can regulate the temperature of the water, or other fluid, to the user's approximate body temperature. This temperature regulation can make the fluid much more comfortable for the user when it is directed into the ear. Furthermore, while embodiments of this specification use water as the delamination fluid, this should be understood to encompass any suitable fluid. For example, purified or deionized water could be used, or an aqueous solution of hydrogen peroxide. In another example, water that has been conditioned with sodium chloride (salt) to an isotonic concentration may also be used. In other embodiments, a more viscous fluid may be used, if it is desirable or necessary to use a more viscous fluid to provide greater force behind the vortical stream of fluid.

There are also disclosed herein various embodiments of the handheld unit, and of the disposable earpiece.

For example, in the FIGURES below, two illustrative shapes are provided for the handheld unit. These shapes should be understood to be nonlimiting and are provided by way of illustrative example only.

Similarly, a number of embodiments of a disposable earpiece are provided. These are also provided by way of illustrative and nonlimiting example. One feature of the disposable earpiece in at least some embodiments of the present specification is that the disposable earpiece includes a fluid guide to impart a vortical motion to the fluid that is used for delamination and removal. This vortical fluid guide can take several forms. For example, in some embodiments, the disposable earpiece has a large ingress aperture and a large egress aperture, with helical fluid guides along the inner edges of the earpiece. This provides relatively more volume, but less directed impulse of the fluid.

In another embodiment, a plurality of ingress ports and egress ports are provided around the aperture of the earpiece. The ingress ports fluidly couple to water channels that terminate at the egress ports. Various embodiments of these water channels are illustrated, such as helical water channels, and linear water channels that terminate in hooked ends. Furthermore, the fluid guides, ingress ports, and egress ports could have a uniform circumference throughout, or could be tapered. For example, the ingress ports may have a larger diameter than the egress ports. This may increase the speed and pressure of the fluid at the egress port relative to the speed and pressure at the ingress port for the same volume of water. The additional velocity and pressure of the water can help to provide the delaminating force. Furthermore, in some embodiments, slight “ramps” are provided at the terminals of the egress ports. This further helps to induce the vortical action in the fluid stream.

In some embodiments, the handheld unit also includes a video capability that can provide a video feedback to the control unit. The control unit could then stream the video, such as via Wi-Fi or Bluetooth, to a handheld device such as a tablet, a smartphone, or a desktop computer. This helps the operator to get a visual indication of the success of the delamination and cerumen removal procedure.

In some cases, the use of the streaming video feed can actually make the device more easily operable for an individual who wishes to use the device at home to remove an impaction. Because the disposable earpiece is designed to not insert farther into the ear canal than is safe, a home user could use the cerumen removal system without fear of damaging the eardrum. With a video feed, the user could see the result of the operation.

In some cases, the handheld unit could also include an aperture through which the operator can see into the ear canal without the aid of a video stream. This could also optionally include a magnifier that can magnify the image of the inner ear canal.

In some cases, the magnifier is hinged so that the operator can move the magnifying glass out of the way, and then have an open aperture into the ear canal. This is useful in cases where the delamination and removal is a first step in a more comprehensive treatment operation. In that case, the medical professional may need to have access to the ear canal with other instruments to perform other work.

A system and method for providing vortical ear irrigation will now be described with more particular reference to the attached FIGURES. It should be noted that throughout the FIGURES, certain reference numerals may be repeated to indicate that a particular device or block is referenced multiple times across several FIGURES. In other cases, similar elements may be given new numbers in different FIGURES. Neither of these practices is intended to require a particular relationship between the various embodiments disclosed. In certain examples, a genus or class of elements may be referred to by a reference numeral (“widget 10”), while individual species or examples of the element may be referred to by a hyphenated numeral (“first specific widget 10-1” and “second specific widget 10-2”).

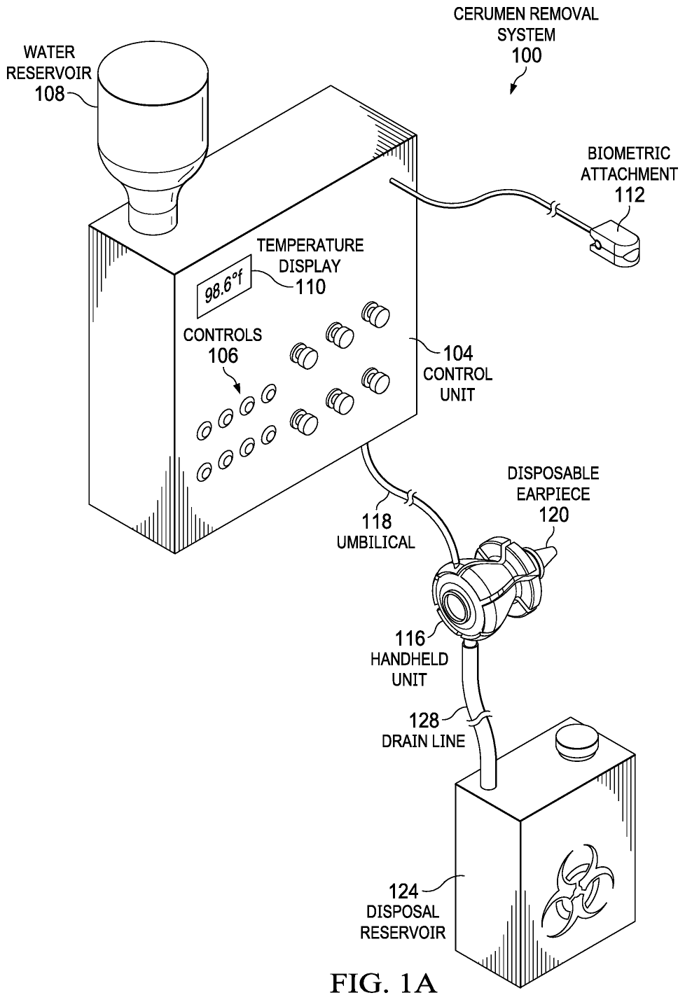

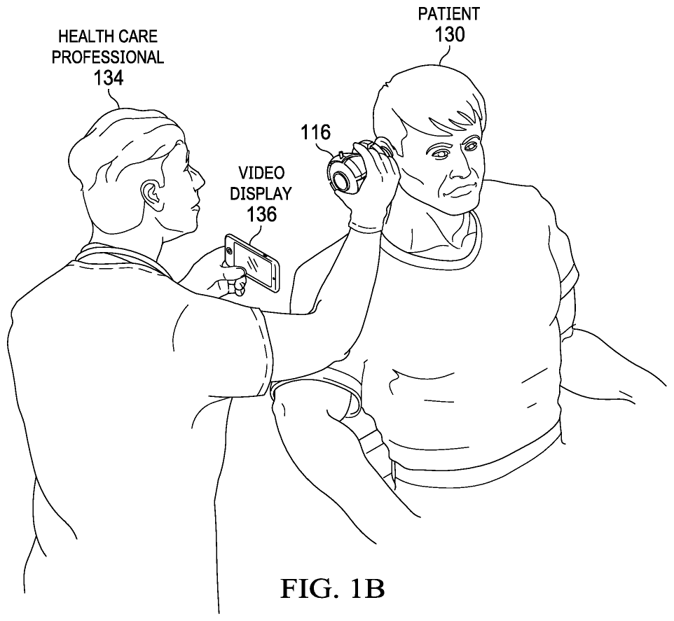

FIG. 1A is a perspective view illustrating selected features of a cerumen removal system 100. In this example, cerumen removal system 100 includes a control unit 104, fluidly and communicatively coupled to a handheld unit 116, via an umbilical 118. Handheld unit 116 is also fluidly coupled to a disposal reservoir 124, via drain line 128. Also in this embodiment, a biometric attachment 112 is communicatively coupled to control unit 104.

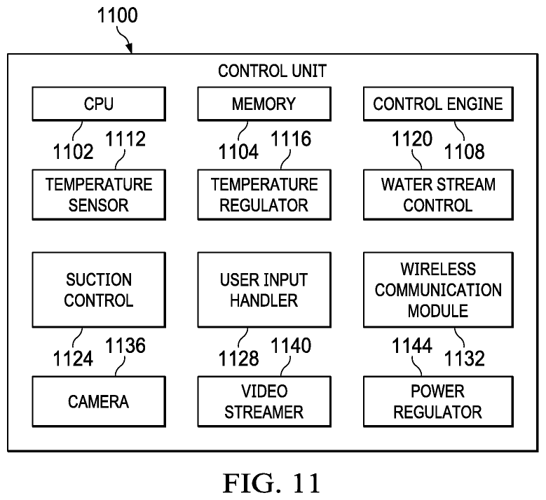

Control unit 104 may include the logic, processing, and control features of cerumen removal system 100. For example, control unit 104 may include a computer control that centrally controls the functions of cerumen removal system 100. Additional features of an embodiment of control unit 104 are shown in FIG. 11, hereafter.

Control unit 104 includes user controls 106 that can be used to control the various functions of cerumen removal system 100, such as switching between vortical and linear water dispersion, switching between heated and unheated fluid, switching on and off a video feed, selecting suction intensity, or other control functions.

In this illustration, a temperature display 110 may also receive a temperature reading from biometric attachment 112. Biometric attachment 112 could be a temperature sensor that reads the patient's temperature, such as via a finger sensor in this illustration. Any other suitable temperature sensor could be used to read the patient's temperature, including one with preset capabilities. Temperature display 110 displays the measured temperature by biometric attachment 112. This can be used, for example, if cerumen removal system 100 is to disperse fluid that is to approximately match the user's temperature for comfort purposes. Temperature display 110 can also be used in the diagnosis of the patient. For example, the medical professional can immediately see whether the patient has a fever. Biometric attachment 112 could also provide additional information, such as the patient's pulse, blood oxygen level, blood pressure, or other biometric indicators. Thus, temperature display 110 could be expanded in some embodiments to provide a more full-featured display that provides a comprehensive view of common health indicators.

In this embodiment, a water reservoir 108 is provided to feed water to handheld unit 116 via control unit 104. Water reservoir 108 could be gravity fed as in this illustration, could be pump fed, or could be provided via any other suitable mechanism. As discussed above, water is used as a representative illustration of a popular fluid choice for the delamination fluid. Other fluids could be used, including more viscous fluids, or water compounds such as saltwater with a salt concentration to an isotonic level. Isotonic saltwater can improve both the comfort and the safety of the user.

Umbilical 118 communicatively and fluidly couples control unit 104 to handheld unit 116. For example, umbilical 118 may include a fluid line to carry fluid from water reservoir 108 to handheld unit 116. Umbilical 118 could also carry communication lines and power lines to handheld unit 116, as necessary and appropriate. This embodiment anticipates a case where there is wired communication between handled unit 116 and control unit 104. However, wireless communication such as via Bluetooth, Wi-Fi, radio frequency (RF), or some other wireless communication means could also be provided.

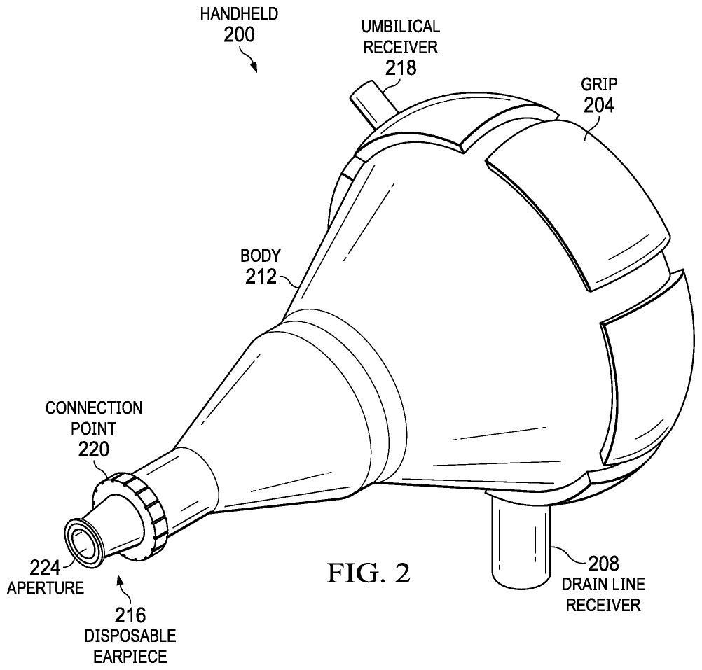

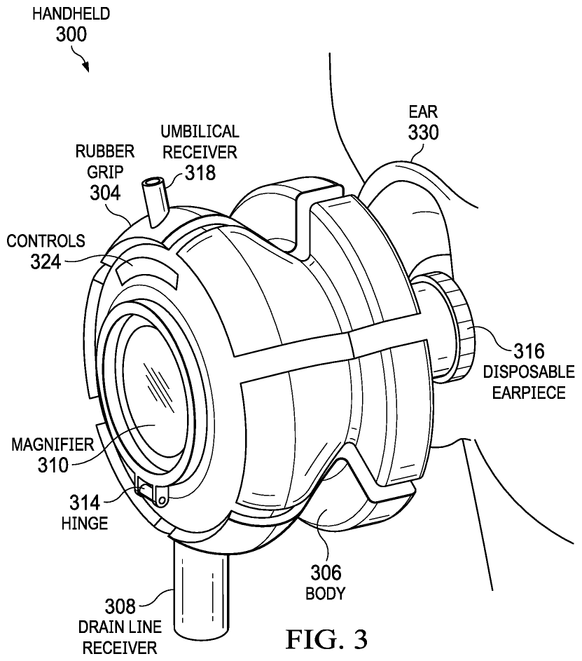

Handheld unit 116 is described in greater detail, for example, in FIGS. 2 and 3 below. In this illustration, it is noted that handheld unit 116 includes a disposable earpiece 120.

Handheld unit 116 is only one illustrative example of an extension that could be used to interface with a human subject. For example, the extension could be fixed to a mobile or stationary unit, or the extension could be affixed to a headset. When the extension is in a fixed position, the unit may be positioned by an operator or the patient. The extension could also include a sensor to detect contact, and to detect when contact is broken. This solution could be particularly useful in a home use scenario, where there is not separate operator.

Handheld unit 116 includes a disposable earpiece 120. Embodiments of disposable earpieces are disclosed in greater detail below.

Once cerumen has been delaminated from the ear canal, handheld unit 116 can switch to a second mode of operation. In this mode, instead of vortical or helical fluid motion, direct or linear fluid may be injected to help break up the cerumen mass.

Drain line 128 fluidly couples handheld unit 116 to disposal reservoir 124. Drain line 128 may carry excess and waste water from handheld unit 116 to disposal reservoir 124. Drain line 128 can also carry debris and other matter removed from the ear canal from handheld unit 116 to disposal reservoir 124. In some cases, disposable earpiece 120 includes a separate egress port that couples to drain line 128 to carry extracted cerumen, waste fluid, and other debris away from the ear. The egress port may provide suction to pull out the waste, or a non-suction solution may be used. For example, in some cases, the movement of the fluid will help displace debris until it falls into the egress port.

In this illustration, disposal reservoir 124 is marked with a bio waste or biohazard marker, which indicates that it should be handled with appropriate safety protocols for biological waste material. This is to help ensure that there is not cross-contamination between various users.

In some embodiments, umbilical 118 may provide constant positive pressure into handheld unit 116 to ensure that waste fluid and debris do not backfill into umbilical 118, or into control unit 104. This helps to ensure that there is not cross-contamination between patients. Furthermore, earpiece 120 is illustrated here as a disposable earpiece intended, for example, for one-time use. This also helps to prevent the spread of infection. In some cases, drain line 128 could also be disposable. In other cases, drain line 128 could be made of a material that is suitable for sanitizing, including high temperature sanitization. Similarly, handheld unit 116 may not be disposable, and may instead be made of a material that can be sanitized, such as via chemical and/or high temperature sanitization. Illustrative materials for handheld unit 116 include specialized metal alloys, stainless steel, ceramics, thermoplastics, and other composite materials by way of nonlimiting example.

In other embodiments, the entire handheld unit 116 could be a one-time-use disposable unit. Alternatively, where electronics are provided, the non-disposable portion of handheld unit 116 may include just the electronics, while all portions of handheld unit 116 through which debris and waste flow may be disposable.

FIG. 1B is a perspective view illustrating a healthcare professional 134 treating a patient 130. In this illustration, healthcare professional 134 is operating a handheld unit 116 of a cerumen removal system 100. For simplicity of the illustration, only the handheld unit 116 is shown in this illustration. However, handheld unit 116 could be connected via an umbilical 118 and a drain line 128 to a control unit 104 and a disposal reservoir 124, respectively.

In this illustration, handheld unit 116 includes a camera feed. For example, handheld unit 116 may have a fiber optic camera, with a fiber-optic cable connecting back to control unit 104. Alternatively, handheld unit 116 could provide video processing, and could further provide a video feed via data lines back to control unit 104.

Either handheld unit 116 or control unit 104 can then stream live video to video display 136. Video display 136 could be, by way of illustrative and nonlimiting example, a cell phone, a tablet, a desktop computer, a dedicated display device that attaches wirelessly or via wires to control unit 104, or any other suitable video display. This allows healthcare professional 134 to view, in real time, the ear canal of patient 130. The healthcare professional 134 can then assess the effectiveness of the cerumen removal, and may perform other work, as necessary.

FIG. 2 is a perspective view illustrating an embodiment of a handheld unit. In this embodiment, handheld unit 200 is visibly different from handheld unit 116 of FIG. 1. This is to illustrate that there are a number of options for the design and structure of handheld unit 200.

In this illustration, handheld unit 200 includes an umbilical receiver 218. Umbilical receiver 218 may include connections to couple to an umbilical, such as umbilical 118 of FIG. 1. Similarly, drain line receiver 200 may include connections to a drain line, such as drain line 128 of FIG. 1. In some cases, drain line receiver 208 may be a simple port or stub to receive a host, while umbilical receiver 218 could be configured to receive either simple fluid via a hose, or a more structured connection. For example, umbilical receiver 218 could receive fluid lines, communication lines, fiber-optic lines, and other connections. In those cases, umbilical receiver 218 may have connection means to ensure that umbilical receiver 218 receives the umbilical in the correct orientation. For example, umbilical receiver 218 could be keyed, or have other mechanisms to ensure that the connection is made correctly.

A body 212 provides the exterior structure of handheld unit 200. Body 212 could be made from molded plastic, molded aluminum, stainless steel, carbon fiber, or some other suitable material.

Grip 204 is provided for a user or a healthcare professional to grip handheld 200. Grip 204 may include, for example, a rubber grip, or some other suitable gripping material. In some cases, a material such as rubber is chosen, to provide better friction and a more secure grip for the operator.

The overall circumference of handheld 200, particularly at the grip point, may be selected for a comfortable grip for an average user, and may also be selected so as not to be too large for smaller operators (e.g., in particular, women or others with smaller hands) and also to not be too small for the largest operators (e.g., large men).

In this case, body 212 tapers down to a connection point 220. Connection point 220 includes mechanical, fluid, and/or electrical connections to engage disposable earpiece 216. In this illustration, disposable earpiece 216 includes an aperture 224, through which the ear canal can be viewed, and through which, in some embodiments, fluid may flow. As shown in the following illustrations, some designs include the option for fluid to flow through aperture 224, while other designs use separate egress ports.

Drain line receiver 208 receives waste fluid and debris, and directs waste fluid and debris to a disposal reservoir such as disposal reservoir 124 of FIG. 1. Depending on the embodiment, drain line receiver 208 could be connected directly to disposable earpiece 216. In this configuration, all waste and debris flow through the disposable part of handheld 200, thus ensuring that waste and debris do not contaminate the nondisposable part. However, even in cases where drain line receiver 208 is connected directly to disposable earpiece 216, it may still be necessary to decontaminate and sterilize handheld 200, to ensure compliance with medical safety regulations.

FIG. 3 is a perspective view illustration of an additional embodiment of a handheld unit. Although handheld 300 is functionally similar to handheld 200 of FIG. 2, handheld 300 is visually and structurally different from handheld 200 of FIG. 2.

Handheld 300 includes an umbilical receiver 318, similar to umbilical receiver 218 of FIG. 2. Handheld 300 also includes a drain line receiver 308, which is similar to drain line receiver 208 of FIG. 2.

Handheld 300 includes a body 306 that is functionally and visually different from body 212 of FIG. 2. In this case, the tapered portion of body 306 can be used as a grip, as illustrated in FIG. 1B. Disposable earpiece 316 is shown in this illustration engaging with the ear of a subject.

Handheld 300 in this case also includes controls 324, which can include both controls and displays. This can enable the operator to manipulate the functions of a cerumen removal system, directly from handheld 300. For example, this could include controls to switch between vortical and linear water injection, water injection and suction, heated and unheated fluid, and other options.

Also in this embodiment, a magnifier 310 covers an aperture of handheld 300. In this case, magnifier 310 is hinged at hinge 314. Thus, magnifier 310 can be flipped down from its closed position, so that there is open access into the subject's ear canal. This can be beneficial to enable the operator to view the ear canal through magnifier 310, and then flip down magnifier 310 to access the ear canal with instruments such as scrapers, suckers, or other instruments that may need to be used to engage in further work on the ear canal. This can be useful if there is to be supplemental operation on the ear canal, in addition to operation of the cerumen removal system illustrated herein.

In some embodiments, handheld 300 directs fluid via umbilical receiver 318 into disposable earpiece 316. This can be directed in different ways. For example, in one illustrative method, fluid is first injected with a vortical orientation to delaminate the impaction from the walls of the ear canal. This may provide some breaking up of the impaction, but advantageously loosens the impaction so that it is easier to remove. Optionally, a somewhat high-pressure, steady stream of water can then be injected to break up the impaction into smaller pieces that are easier to dispose of. Finally, suction can be applied to suck the broken-up impaction and the wastewater out into a drain line via drain line receiver 308.

Advantageously, this can be done with heated fluid that is heated to the user's approximate body temperature, and that is also optionally isotonic. This can greatly increase the comfort of the subject, while providing effective removal of the impaction.

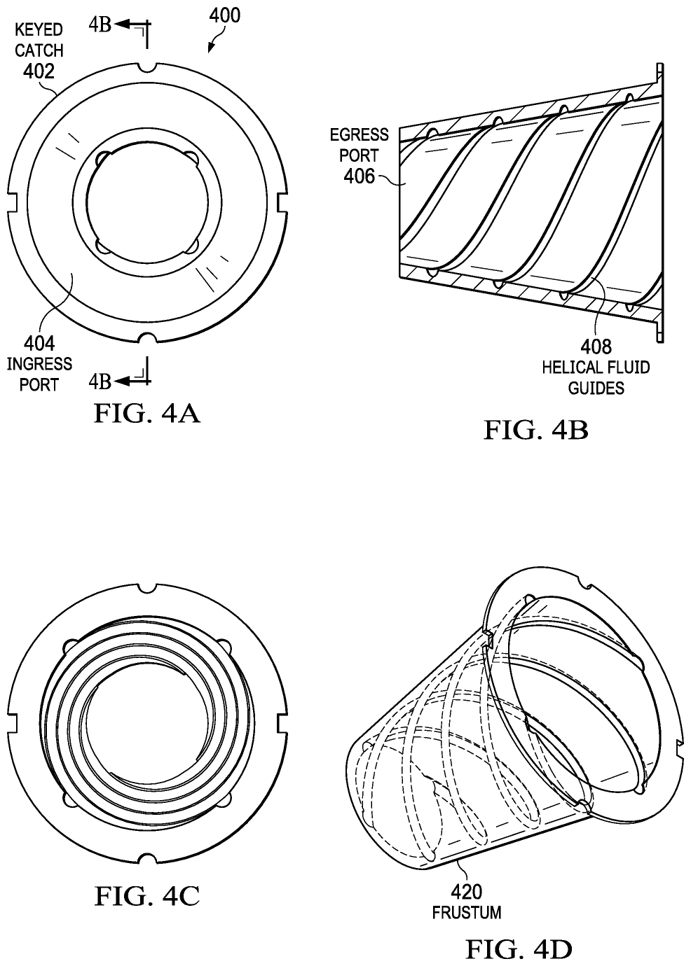

FIGS. 4A, 4B, 4C, and 4D illustrate one embodiment of a disposable earpiece. Several embodiments of disposable earpieces are illustrated throughout this specification, and these should be understood to be nonlimiting and illustrative examples.

Disposable earpiece 400 is designed to engage the handheld with a keyed catch mechanism 402. A keyed catch 402 is provided as one illustrative and nonlimiting example, only. Other embodiments could include a screw-on operation, a pressure coupling, or any other suitable coupling mechanism.

Disposable earpiece 400 is a truncated cone configuration, having a single large ingress port 404 at the base of the cone. This large ingress port may vary in size, but may be optimized for maximum fluid flow. For example, the ingress port may occupy at least approximately 50% of the area of the base of the cone. In other embodiments, the ingress port occupies at least approximately 75% of the area of the base of the cone. In yet another embodiment, the ingress port occupies at least approximately 90% of the area of the base of the cone.

A single large egress port 406 is provided at the frustum 420 of the truncated cone. The egress port may occupy at least approximately 50% of the area of the frustum. In other embodiments, the egress port occupies at least approximately 75% of the area of the frustum. In yet another embodiment, the egress port occupies at least approximately 90% of the area of the frustum.

Helical fluid guides 408 are, in this embodiment, etched into the inside wall of disposable earpiece 400. These terminate at egress port 406.

When a volume of water is injected into disposable earpiece 400 via ingress port 404, some of the water will be forced into helical fluid guides 408, and exit with a vortical motion. The remainder of the fluid will experience less vortical motion, but may flow in a more linear fashion through disposable earpiece 400.

This illustrates a combination embodiment, where vortical and linear motion are imparted to separate parts of the fluid stream at the same time. The vortical motion may help to delaminate the impaction, while the linear motion can help to break up the impaction. However, this joint action comes with a trade-off. Both the linear and the vortical fluid streams may be less intense than if they were provided separately. This embodiment is advantageous for use cases where a less intense vortical and linear stream are desired.

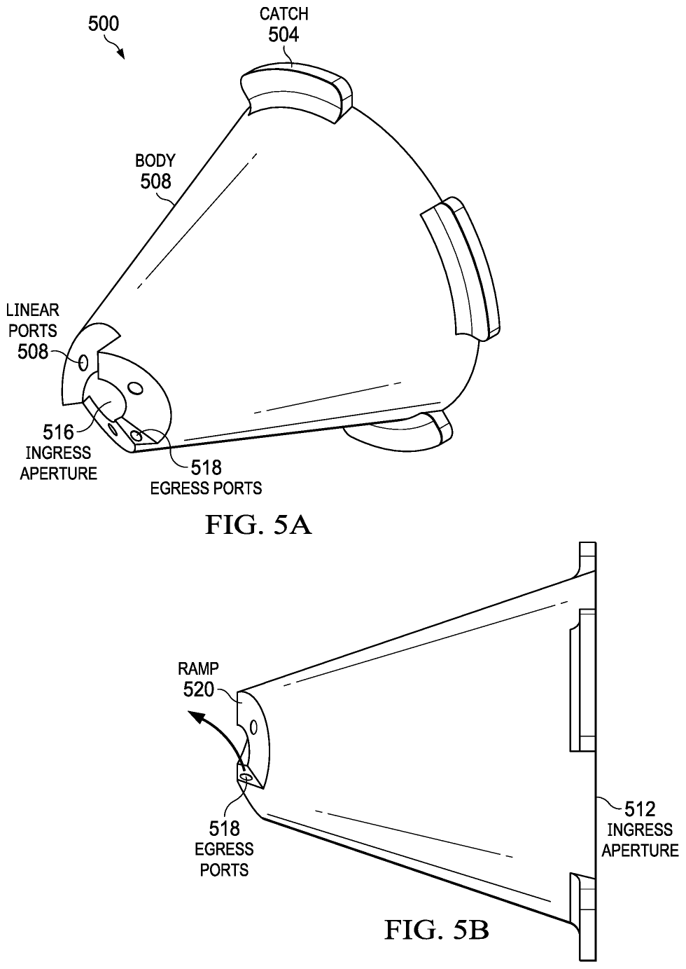

FIGS. 5A and 5B provide a perspective view of another embodiment of a disposable earpiece. Disposable earpiece 500 includes a catch 504 to securely fasten to the handheld unit. As with other earpieces, this is provided as an illustrative example only.

Disposable earpiece 500 also includes a body 508. Body 508 is a truncated cone, but in this case, it does not have a flat frustum. Rather, the frustum is molded into a series of ramps 520. Ramps 520 help to launch water coming out of egress ports 518, to impart additional vortical motion to the water.

As in other embodiments, an ingress aperture 512 and egress aperture 516 are provided. In this case, ingress aperture 512 and egress aperture 516 are open apertures, and in at least some embodiments, it is not intended for fluid to flow from ingress aperture 512 to egress aperture 516. Rather, separate ingress ports on the base of disposable earpiece 500 match up to egress ports 518, and provide fluid to egress ports 518.

Optionally, earpiece 500 may have a large central aperture, although unlike the embodiment of FIG. 4A, the central aperture does not also serve as the ingress port to the vortical fluid guide. The size constraints for the central aperture as described above are applicable, and the other embodiments disclosed herein (e.g., those with separate fluid guides) may similarly have large central apertures.

Also illustrated in this embodiment are linear ports 520. Linear ports 520 may also have their own ingress ports, and provide a different type of water motion. Whereas egress ports 518 may be fluidly connected to helical or hooked fluid guides, linear ports 520 may have directly linear fluid guides. This can allow for switching between different modes. Egress ports 518 provide vortical motion that can be used to delaminate an impaction from the ear canal. The apparatus can then be switched to a linear mode, and linear ports 520 impart linear water that can be used to help break up the ball of wax.

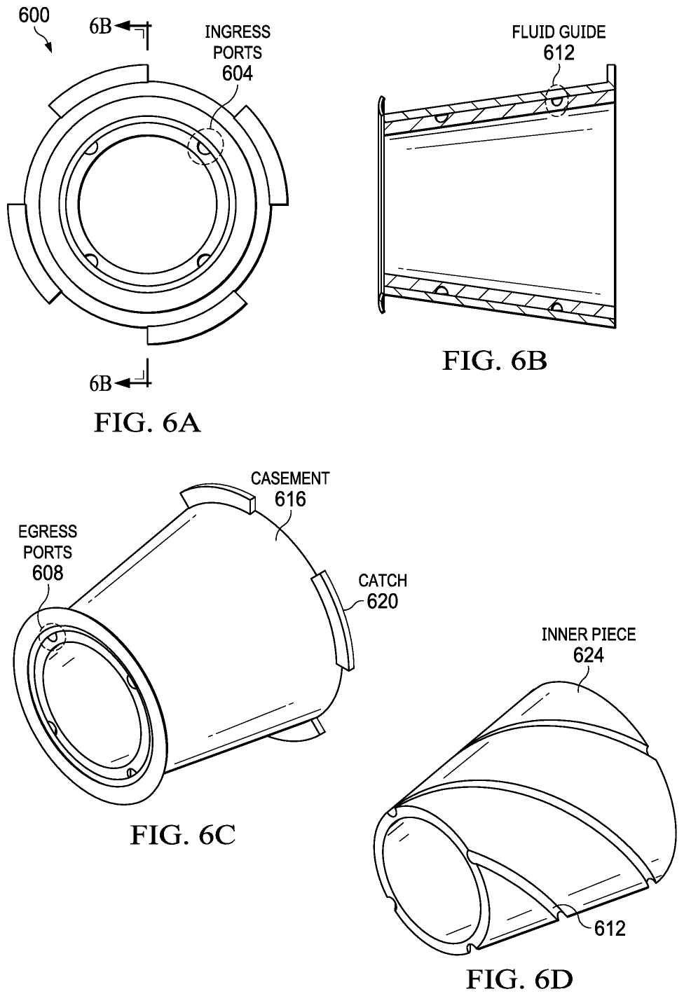

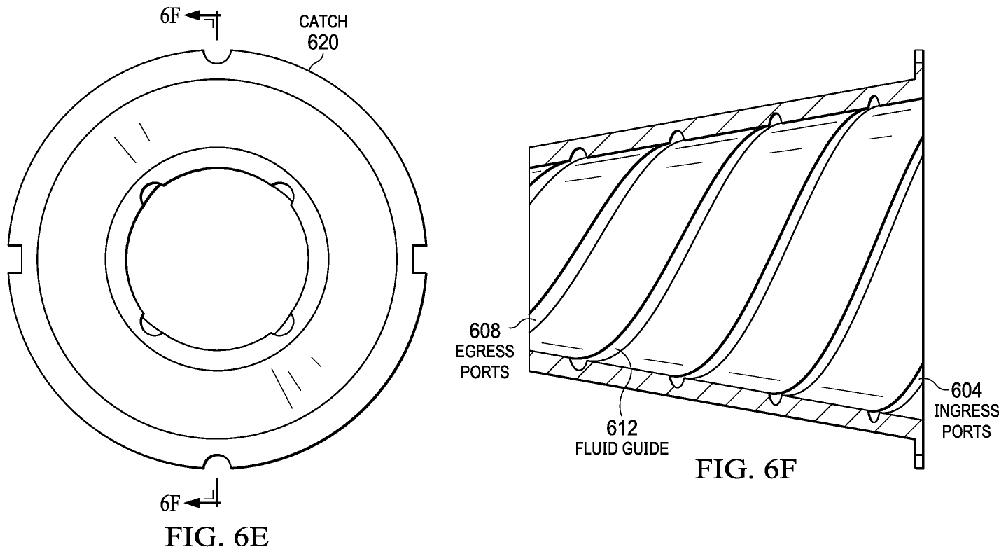

FIGS. 6A, 6B, 6C, 6D, 6E, and 6F illustrate yet another embodiment of a disposable earpiece. In this embodiment, ingress ports 604 are visible, along with egress ports 608. Fluid guides 612 are helical, and connect ingress ports 604 to egress ports 608. Helical fluid guides 612 impart a vortical motion to water flowing through them, and thus water exiting at egress ports 608 exits in a vortical fashion.

Disposable earpiece 600 illustrates an embodiment with a two-piece construction. For example, comparing disposable earpiece 400 to disposable earpiece 500 and disposable earpiece 600, it can be seen that disposable earpiece 400 would be easier to manufacture as a single piece, such as via injection molding. Because disposable earpiece 500 of FIG. 5 includes internal fluid guides, it would be more difficult to manufacture any single piece via injection molding.

Disposable earpiece 600 of FIG. 6 illustrates one manufacturing option. In this case, disposable earpiece 600 can be manufactured in two pieces. An outer casement 616 includes catches 620, and tightly engages an inner piece 624. Outer casement 616 and inner piece 624 can be manufactured separately, and then inner piece 624 can be inserted into casement 616. This provides internal fluid guides 612, which in this configuration are helical, although linear fluid guides could also be provided in the case where there is both vortical and linear fluid motion desired.

Once casement 616 and inner piece 624 are manufactured separately, they could be affixed via an adhesive, or via mechanical coupling if they are manufactured with tight tolerances. In other cases, they could have a one-way latch that engages, but that does not readily disengage.

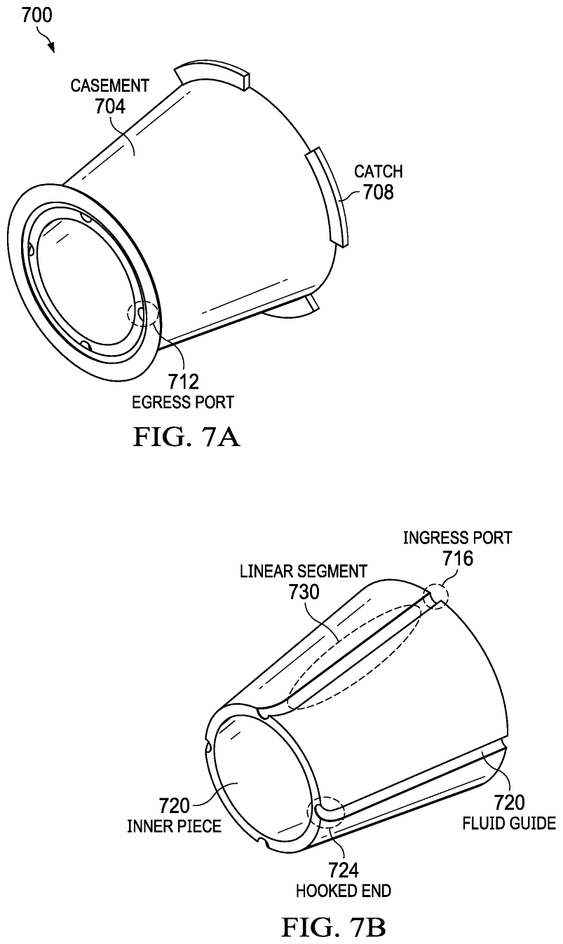

FIGS. 7A and 7B are a perspective view illustration of a further embodiment of a disposable earpiece. In this case, disposable earpiece 700 once again is shown as a truncated cone with a flat frustum. Similar to disposable earpiece 600 of FIG. 6, disposable earpiece 700 of FIG. 7 is illustrated as a two-piece construction. Specifically, a casement 704 includes catches 708, while an inner piece 720 includes a number of fluid guides. Ingress ports 716 and egress ports 712 provide ingress and egress for the fluid, while the ingress and egress apertures are open, and no fluid is intended to pass through them.

Disposable earpiece 700 illustrates another option for fluid guide 720. In this case, rather than provide a helical fluid guide to impart vortical motion to the fluid, fluid guide 720 includes a linear segment 730 terminated at a hooked end 724. This helps to impart maximum linear velocity to fluid as it travels down linear segment 730, and then hooked end 724 redirects the water in a helical direction, thus providing the helical motion at egress port 712.

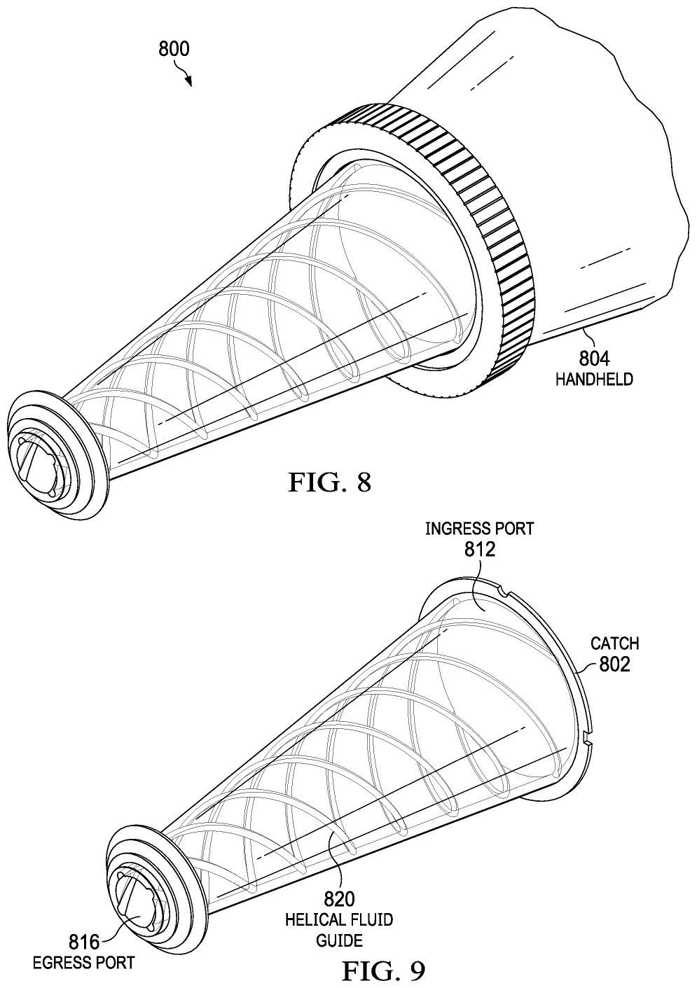

FIGS. 8 and 9 provide a block diagram illustration of another further embodiment of a disposable earpiece. In this case, disposable earpiece 800 may be manufactured as a single piece, and may engage to handheld 804 via catches 802, may screw on, or may engage via some other means. In this case, the large aperture acts as an ingress port 812, while at a removed end is an egress port 816.

Disposable earpiece 800 is substantially a truncated cone, with ingress port 812 at the base and egress port 816 at a frustum.

A helical fluid guide 820 is provided to impart helical or vortical motion to water entering ingress port 812 and exiting at egress port 816. This embodiment is similar to the embodiment of FIG. 4, where a combination of linear water and vortical water streams may provide both delamination and breakup simultaneously, at the expense of higher pressure for both operations.

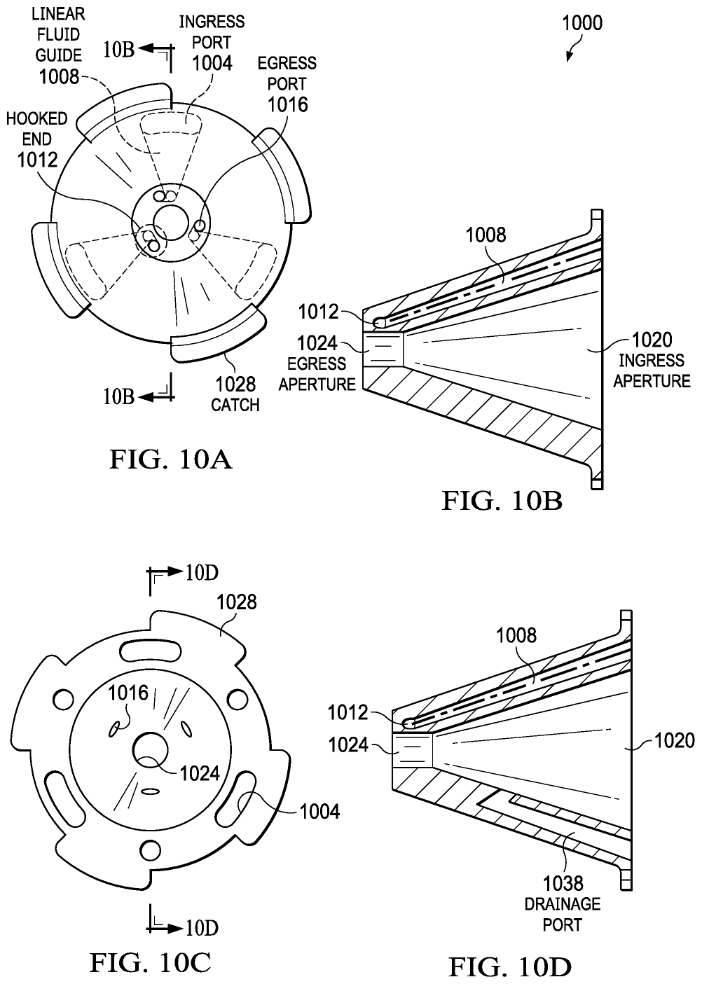

FIGS. 10A, 10B, 10C, and 10D illustrate an additional further embodiment of a disposable earpiece. Disposable earpiece 1000 includes, as before, a catch mechanism 1028 that engages the handheld unit. There is also an ingress aperture 1020 and an egress aperture 1024. As before, it is not intended for fluid to flow into ingress aperture 1020.

In this embodiment, three egress ports 1016 are provided and communicatively coupled to three ingress ports 1004. As illustrated here, ingress ports 1004 are ovular in shape, and are larger in volume than egress ports 1004. A linear fluid guide 1008 tapers down to a hooked end 1012. This allows a relatively larger volume of fluid to flow into ingress port 1004, and to flow out of egress ports 1016 with both a higher speed and higher pressure. Fully linear fluid guides are not illustrated in this embodiment, but fully linear fluid guides to provide linear water for breakup, similar to those illustrated in FIGS. 5A and 5B, could also be provided.

Also seen in this illustration is a drainage port 1038. This illustrates another potential feature. Although fluid is not intended to flow from ingress aperture 1020 into egress aperture 1024, egress aperture 1024 is open. Wastewater and debris may flow into egress aperture 1024. If suction is engaged, then the wastewater and debris may flow into drainage port 1038. Drainage port 1038 has a hooked end, so that fluid does not flow back out from drainage port 1038 into aperture 1020. Drainage port 1038 may fluidly couple to a drain line and ultimately to a disposal reservoir, such as disposal reservoir 124 of FIG. 1.

Some embodiments may include a replaceable tubular flexible tip, which may provide different spray patters such as circular or fan spray patterns. These could be provided as extensions to any of the earpieces discussed above, or they could be provided as separate extensions, which provide non-vortical or non-helical spray patterns. In some cases, disposable tips could be switched out to provide various spray patterns (e.g., vortical, direct, circular, fan, etc.).

FIG. 11 is a block diagram illustration of a control unit 1100. Control unit 1100 may be an embodiment, for example, of control unit 104 of FIG. 1.

Control unit 1100 includes a CPU 1102 and a memory 1104. CPU 1102 may comprise any suitable number of processor cores and supporting logic. In particular embodiments, a CPU 1102 is embodied within a socket that is permanently or removably coupled to control unit 1100.

Memory 1104 may comprise any form of volatile or non-volatile memory including, without limitation, magnetic media (e.g., one or more tape drives), optical media, random access memory (RAM), read-only memory (ROM), flash memory, removable media, or any other suitable local or remote memory component or components. Memory 1104 may be used for short, medium, and/or long-term storage by control unit 1100. Memory 1104 may store any suitable data or information, including software embedded in a computer-readable medium, and/or encoded logic incorporated in hardware or otherwise stored (e.g., firmware). Memory 1104 may store data that is used by cores of CPU 1102. In some embodiments, memory 1104 may also comprise storage for instructions that may be executed by the cores of CPU 1102 or other processing elements to provide functionality associated with the control engine 1108, or other components of control unit 1100.

In various embodiments, a control engine 1108 operates as an out-of-band asynchronous compute agent which is capable of interfacing with the various elements of control unit 1100 to collect telemetry data with no or minimal disruption to running processes on CPU 1102. For example, control engine 1108 may comprise a dedicated processing element (e.g., a processor, controller, or other logic), which provides the functionality of control engine 1108 (e.g., by executing software instructions), thus conserving processing cycles of CPU 1102 for operations associated with the workloads performed by control unit 1100. Moreover, control engine 1108 may operate asynchronously with respect to CPU 1102 and may gather at least some of the telemetry data without increasing the load on CPU 1102.

Control engine 1108 may operate in hardware, firmware, and/or software, such as including instructions stored in memory 1104 that when executed instruct CPU 1102 to provide the functions of the control engine. Control engine 1108 provides switching control between the various modes of control unit 1100, and also controls other functions. Water stream control 1120 may be provided to control different modes, such as linear water stream mode and vortical water stream mode.

Temperature regulator 1116 can regulate the temperature by heating water in a water reservoir, or stopping heating when the heating is complete.

Temperature sensor 1112 may sense the subject's temperature, and may be used by temperature regulator 1116 to determine what temperature to heat the fluid to.

Suction control 1124 controls suction functions, such as is illustrated in FIG. 10D, where suction is used in drainage port 1038 to suck out wastewater and debris.

Power regulator 1144 provides and regulates power to different system components.

Camera 1136 receives a live camera feed from a camera, such as a fiber optic camera, embedded in the handheld or in the disposable earpiece.

Video streamer 1140 uses a network interface, such as a wireless network interface, to stream live video from camera 1136 so that it can be viewed on a device such as a smart phone, a tablet, a desktop computer, or a dedicated video viewing device.

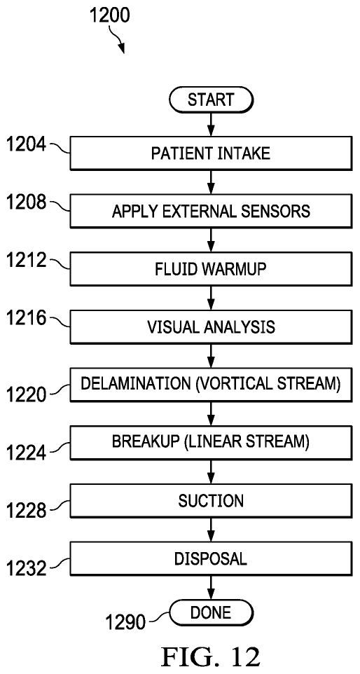

FIG. 12 is a flowchart of a method 1200 of performing cerumen impaction removal with a cerumen removal system of the present specification.

In block 1204, patient intake may occur to receive necessary information about the patient, to receive necessary permissions and legal compliance, to set up billing, and to otherwise prepare the patient for the procedure.

In block 1208, external sensors may be applied to the patient. This could include a temperature sensor, a heart rate sensor, a blood saturation sensor, a blood oxygen level sensor, and a blood pressure cuff, all by way of nonlimiting and illustrative example.

In block 1212, the apparatus performs fluid warmup. This is to warm up the water or other fluid to approximate the patient's temperature within a threshold such as one or two degrees, to help ensure the comfort and safety of the patient.

In block 1216, the provider can perform visual analysis, such as by looking through a magnifier, or viewing the live video. In this way, the provider can assess the nature and severity of the impaction, and determine a treatment option.

In block 1220, delamination occurs. For example, the provider sets the control unit to provide a vortical stream of fluid that is used to delaminate the impaction from the user's ear canal.

In block 1224, the operator may switch to a linear stream to break up the impaction into smaller pieces that are easy to pull out.

In block 1228, the operator may engage suction to suck out the wastewater and debris generated from the delamination and breakup procedures.

In block 1232, the operator disposes of the waste matter.

In block 1290, the method is done.

The foregoing outlines features of several embodiments so that those skilled in the art may better understand various aspects of the present disclosure. The embodiments disclosed can readily be used as the basis for designing or modifying other processes and structures to carry out the teachings of the present specification. Any equivalent constructions to those disclosed do not depart from the spirit and scope of the present disclosure. Design considerations may result in substitute arrangements, design choices, device possibilities, hardware configurations, software implementations, and equipment options.

As used throughout this specification, a “memory” is expressly intended to include both a volatile memory and a non-volatile memory. Thus, for example, an “engine” as described above could include instructions encoded within a memory that, when executed, instruct a processor to perform the operations of any of the methods or procedures disclosed herein. It is expressly intended that this configuration reads on a computing apparatus “sitting on a shelf” in a non-operational state. For example, in this example, the “memory” could include one or more tangible, non-transitory computer-readable storage media that contain stored instructions. These instructions, in conjunction with the hardware platform (including a processor) on which they are stored may constitute a computing apparatus.

In other embodiments, a computing apparatus may also read on an operating device. For example, in this configuration, the “memory” could include a volatile or run-time memory (e.g., RAM), where instructions have already been loaded. These instructions, when fetched by the processor and executed, may provide methods or procedures as described herein.

In yet another embodiment, there may be one or more tangible, non-transitory computer-readable storage media having stored thereon executable instructions that, when executed, cause a hardware platform or other computing system, to carry out a method or procedure. For example, the instructions could be executable object code, including software instructions executable by a processor. The one or more tangible, non-transitory computer-readable storage media could include, by way of illustrative and non-limiting example, a magnetic media (e.g., hard drive), a flash memory, a read-only memory (ROM), optical media (e.g., CD, DVD, Blu-Ray), non-volatile random access memory (NVRAM), non-volatile memory (NVM) (e.g., Intel 3D Xpoint), or other non-transitory memory.

There are also provided herein certain methods, illustrated for example in flow charts and/or signal flow diagrams. The order or operations disclosed in these methods discloses one illustrative ordering that may be used in some embodiments, but this ordering is no intended to be restrictive, unless expressly stated otherwise. In other embodiments, the operations may be carried out in other logical orders. In general, one operation should be deemed to necessarily precede another only if the first operation provides a result required for the second operation to execute. Furthermore, the sequence of operations itself should be understood to be a non-limiting example. In appropriate embodiments, some operations may be omitted as unnecessary or undesirable. In the same or in different embodiments, other operations not shown may be included in the method to provide additional results.

In certain embodiments, some of the components illustrated herein may be omitted or consolidated. In a general sense, the arrangements depicted in the FIGURES may be more logical in their representations, whereas a physical architecture may include various permutations, combinations, and/or hybrids of these elements.

With the numerous examples provided herein, interaction may be described in terms of two, three, four, or more electrical components. These descriptions are provided for purposes of clarity and example only. Any of the illustrated components, modules, and elements of the FIGURES may be combined in various configurations, all of which fall within the scope of this specification.

In certain cases, it may be easier to describe one or more functionalities by disclosing only selected element. Such elements are selected to illustrate specific information to facilitate the description. The inclusion of an element in the FIGURES is not intended to imply that the element must appear in the invention, as claimed, and the exclusion of certain elements from the FIGURES is not intended to imply that the element is to be excluded from the invention as claimed. Similarly, any methods or flows illustrated herein are provided by way of illustration only. Inclusion or exclusion of operations in such methods or flows should be understood the same as inclusion or exclusion of other elements as described in this paragraph. Where operations are illustrated in a particular order, the order is a nonlimiting example only. Unless expressly specified, the order of operations may be altered to suit a particular embodiment.

Other changes, substitutions, variations, alterations, and modifications will be apparent to those skilled in the art. All such changes, substitutions, variations, alterations, and modifications fall within the scope of this specification.

In order to aid the United States Patent and Trademark Office (USPTO) and, any readers of any patent or publication flowing from this specification, the Applicant: (a) does not intend any of the appended claims to invoke paragraph (f) of 35 U.S.C. section 112, or its equivalent, as it exists on the date of the filing hereof unless the words “means for” or “steps for” are specifically used in the particular claims; and (b) does not intend, by any statement in the specification, to limit this disclosure in any way that is not otherwise expressly reflected in the appended claims, as originally presented or as amended.

### Example Implementations

There is disclosed in one example an ear irrigation system, comprising: a control unit comprising a fluid reservoir; a disposable earpiece constructed to interface with a human subject's ear canal; an extension unit configured to receive the disposable earpiece and to impart fluid to an ingress port of the disposable earpiece; and an umbilical to fluidly couple the fluid reservoir to the extension unit; wherein the control unit is programmed to, responsive to an input from an operator, pump fluid from the fluid reservoir to the extension unit; and wherein the disposable earpiece comprises a plurality of vortical fluid guides to guide fluid from an ingress port to an egress port, and to impart to the fluid at the egress port a vortical motion to delaminate a cerumen impaction from the human user's ear canal.

There is further disclosed an example ear irrigation system, further comprising a view port disposed within the extension unit to enable viewing of the subject's ear canal.

There is further disclosed an example ear irrigation system, further comprising a magnifying glass over the view port.

There is further disclosed an example ear irrigation system, wherein the magnifying glass is hingedly removable from the view port.

There is further disclosed an example ear irrigation system, further comprising a thermometer to measure the subject's body temperature.

There is further disclosed an example ear irrigation system, further comprising a heater to heat fluid in the reservoir to within a threshold of the subject's measured body temperature.

There is further disclosed an example ear irrigation system, wherein the extension unit further comprises operator controls.

There is further disclosed an example ear irrigation system, wherein the extension unit further comprises a camera disposed to view the subject's ear canal.

There is further disclosed an example ear irrigation system, wherein the control unit further comprises a video display to receive video from the camera.

There is further disclosed an example ear irrigation system, wherein the control unit further comprises a wireless video feed to provide video to a wireless device.

There is further disclosed an example ear irrigation system, further comprising a waste reservoir to receive waste from the extension unit.

There is further disclosed an example ear irrigation system, further comprising a suction control to provide suction at the subject's ear canal.

There is further disclosed an example ear irrigation system, wherein the disposable ear piece further comprises a waste port to receive waste, wherein the waste port is fluidly coupled to the waste reservoir.

There is further disclosed an example ear irrigation system, wherein the extension unit and the disposable earpiece further comprise a non-vortical fluid guide.

There is further disclosed an example ear irrigation system, where the extension unit is a handheld unit.

There is also disclosed an example earpiece for an ear irrigation system, comprising: a truncated conoidal body structure having a conoidal base at a first end, and a frustum at a second end, the frustum sized to interface with a human ear canal; and a fluid guide having an ingress port to receive a volume of fluid, a fluid guide to impart a vortical action to the volume of fluid, and an egress port to direct the volume of fluid into the human ear canal.

There is further disclosed an example earpiece, further comprising a primary central aperture occupying at least approximately 50% of the surface area of the conoidal base.

There is further disclosed an example earpiece, further comprising a primary central aperture occupying at least approximately 75% of the surface area of the conoidal base.

There is further disclosed an example earpiece, further comprising a primary central aperture occupying at least approximately 90% of the surface area of the conoidal base.

There is further disclosed an example earpiece, wherein the primary central aperture is the ingress port.

There is further disclosed an example earpiece, wherein the primary central aperture is separate from the fluid guide.

There is further disclosed an example earpiece, further comprising fastening means to fasten the earpiece to a handheld unit of an ear irrigation system.

There is further disclosed an example earpiece, wherein the fluid guide is a linear-to-hooked fluid guide.

There is further disclosed an example earpiece, wherein the frustum comprises a ramp at a terminal end of the fluid guide.

There is further disclosed an example earpiece, further comprising a plurality of fluid guides.

There is further disclosed an example earpiece, wherein the ingress port has a larger surface area than the egress port.

There is further disclosed an example earpiece, wherein the plurality of fluid guides are disposed within a sidewall of the truncated conoidal body.

There is further disclosed an example earpiece, wherein the fluid guide comprises a plurality of vortical fluid guides disposed within a sidewall of the truncated conoidal body.

There is further disclosed an example earpiece, wherein the fluid guide comprises a large primary aperture with spiral ribbing disposed along a sidewall.

There is further disclosed an example earpiece, wherein the earpiece is disposable.

There is also disclosed an example method of manufacturing an earpiece for an ear irrigation system, comprising: forming a primary body of a polymer material into a conoidal shape; forming attachment means to a base of the primary body; and subtractively forming a plurality of vortical fluid guides within a sidewall of the primary body.

There is further disclosed an example method, further comprising forming a frustum on a removed end from the base, and terminating the vortical fluid guides at the frustum.

There is further disclosed an example method, wherein forming the frustum comprises forming ramps at terminators of the vortical fluid guides.

There is further disclosed an example method, further comprising subtractively forming a large central aperture through the primary body.

There is further disclosed an example method, wherein forming the primary body comprises 3-D printing the primary body.

There is further disclosed an example earpiece manufactured according to the method of a number of the above examples.

There is also disclosed an example method of removing a cerumen impaction from a human subject's ear canal, comprising: applying a vortical fluid stream to the cerumen impaction until the impaction is substantially delaminated from ear canal; and extracting the impaction.

There is further disclosed an example method, further comprising applying a linear fluid stream to the delaminated impaction to break up the impaction.

There is further disclosed an example method, further comprising applying suction to remove the broken-up impaction, and waste fluid.

What is claimed is:

1. An ear irrigation system, comprising:
a control unit comprising a fluid reservoir;
a disposable earpiece constructed to interface with a human subject's ear canal;
an extension unit configured to receive the disposable earpiece and to impart fluid to an ingress port of the disposable earpiece; and
an umbilical to fluidly couple the fluid reservoir to the extension unit;
wherein the control unit is programmed to, responsive to an input from an operator, pump fluid from the fluid reservoir to the extension unit; and
wherein the disposable earpiece comprises a plurality of vortical fluid guides to guide fluid from an ingress port to an egress port, and to impart to the fluid at the egress port a vortical motion to delaminate a cerumen impaction from the human user's ear canal.

2. The ear irrigation system of claim 1, further comprising a view port disposed within the extension unit to enable viewing of the subject's ear canal.

3. The ear irrigation system of claim 2, further comprising a magnifying glass over the view port.

4. The ear irrigation system of claim 3, wherein the magnifying glass is hingedly removable from the view port.

5. The ear irrigation system of claim 1, further comprising a thermometer to measure the subject's body temperature.

6. The ear irrigation system of claim 5, further comprising a heater to heat fluid in the reservoir to within a threshold of the subject's measured body temperature.

7. The ear irrigation system of claim 1, wherein the extension unit further comprises operator controls.

8. The ear irrigation system of claim 1, wherein the extension unit further comprises a camera disposed to view the subject's ear canal.

9. The ear irrigation system of claim 8, wherein the control unit further comprises a video display to receive video from the camera.

10. The ear irrigation system of claim 8, wherein the control unit further comprises a wireless video feed to provide video to a wireless device.

11. The ear irrigation system of claim 1, further comprising a waste reservoir to receive waste from the extension unit.

12. The ear irrigation system of claim 11, further comprising a suction control to provide suction at the subject's ear canal.

13. The ear irrigation system of claim 11, wherein the disposable ear piece further comprises a waste port to receive waste, wherein the waste port is fluidly coupled to the waste reservoir.

14. The ear irrigation system of claim 1, wherein the extension unit and the disposable earpiece further comprise a non-vortical fluid guide.

15. The ear irrigation system of claim 1, wherein the extension unit is a handheld unit.

16. An earpiece for an ear irrigation system, comprising:
a truncated conoidal body structure having a conoidal base at a first end, and a frustum at a second end, the frustum sized to interface with a human ear canal; and
a fluid guide having an ingress port to receive a volume of fluid, a fluid guide to impart a vortical action to the volume of fluid, and an egress port to direct the volume of fluid into the human ear canal.

17. The earpiece of claim 16, further comprising a primary central aperture occupying at least approximately 50% of the surface area of the conoidal base.

18. The earpiece of claim 16, further comprising a primary central aperture occupying at least approximately 75% of the surface area of the conoidal base.

19. The earpiece of claim 16, further comprising a primary central aperture occupying at least approximately 90% of the surface area of the conoidal base.

20. The earpiece of claim 17, wherein the primary central aperture is the ingress port.

21. The earpiece of claim 17, wherein the primary central aperture is separate from the fluid guide.

22. The earpiece of claim 16, further comprising fastening means to fasten the earpiece to a handheld unit of an ear irrigation system.

23. The earpiece of claim 16, wherein the fluid guide is a linear-to-hooked fluid guide.

24. The earpiece of claim 23, wherein the frustum comprises a ramp at a terminal end of the fluid guide.

25. The earpiece of claim 23, further comprising a plurality of fluid guides.

26. The earpiece of claim 25, wherein the ingress port has a larger surface area than the egress port.

27. The earpiece of claim 26, wherein the plurality of fluid guides are disposed within a sidewall of the truncated conoidal body.

28. The earpiece of claim 16, wherein the fluid guide comprises a plurality of vortical fluid guides disposed within a sidewall of the truncated conoidal body.

29. The earpiece of claim 16, wherein the fluid guide comprises a large primary aperture with spiral ribbing disposed along a sidewall.

30. The earpiece of claim 29, wherein the earpiece is disposable.

31. A method of removing a cerumen impaction from a human subject's ear canal, comprising:
applying a vortical fluid stream to the cerumen impaction until the impaction is substantially delaminated from ear canal; and
extracting the impaction.

32. The method of claim 31, further comprising applying a linear fluid stream to the delaminated impaction to break up the impaction.

33. The method of claim 32, further comprising applying suction to remove the broken-up impaction, and waste fluid.
